(19) United States
(12) Patent Application Publication
(10) Pub. No.: US 2013/0295345 A1
(43) Pub. Date: Nov. 7, 2013

(54) MEDICAL ADHESIVE FILM

(71) Applicant: WUHAN VSD MEDICAL SCIENCE & TECHNOLOGY CO.,LTD., Wuhan (CN)

(72) Inventors: Jiuhong SONG, Wuhan (CN); Fuxing PEI, Wuhan (CN); Bo PENG, Wuhan (CN); Hongxia GUAN, Wuhan (CN)

(21) Appl. No.: 13/935,586

(22) Filed: Jul. 5, 2013

Related U.S. Application Data
(63) Continuation-in-part of application No. PCT/CN2011/085086, filed on Dec. 30, 2011.

(30) Foreign Application Priority Data
Jan. 5, 2011 (CN) 201110001116.3

(51) Int. Cl. A61L 24/00

(52) U.S. Cl. CPC A61L 24/001
USPC 428/195.1

(57) ABSTRACT

An adhesive film including an upper surface including a developing reference axis. The adhesive film is simple in structure, accurate in positioning, and convenient for practice.

### CROSS-REFERENCE TO RELATED APPLICATIONS

This application is a continuation-in-part of International Patent Application No. PCT/CN2011/085086 with an international filing date of Dec. 30, 2011, designating the United States, now pending, and further claims priority benefits to Chinese Patent Application No. 201110001116.3 filed Jan. 5, 2011. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 14781 Memorial Drive, Suite 1319, Houston, Tex. 77079.

### BACKGROUND OF THE INVENTION

1. Field of the Invention

The invention relates to the field of medical devices, and more particularly to an adhesive film comprising a developing reference axis.

2. Description of the Related Art

Typical adhesive films include a surgical film, a transparent film, and a sealing film. Each kind of clinical adhesive films has a relative single function, for instance, to protect the surgical environment, to prevent the cross infection of incisions, or to facilitate the operation for medical personals.

In the process of diagnosis, nursing, surgery, and interventional therapy, it is required to position the focus, the objective within the surgical field, and the optical fiber intervention. A typical medical positioning mainly employs the X-ray image, which, however, cannot achieve an accurate positioning of the focus, thus, patients have to take the X-ray image repeatedly, thereby being harmful to the body such as killing a large amount of leukocyte, lowering the immune ability of the body, resulting in diseases, and affecting the rehabilitation after the surgery. An improved method includes positioning, sterilizing, and sticking the adhesive film. However, the focus label on the adhesive film will fade away during sterilization, thereby resulting in inaccurate positioning.

Another improved method is using a developing pen for positioning. However, the developing ink is very easy to remove in the sterilization process or fades away after being diluted by tissue solution and the blood. Besides, the use of the developing pen easily causes the cross infection of the surgical field. Even though a sterilized developing pen is employed to locate the objective within the surgical field and a surgical adhesive film is stuck to protect and fix the developing ink, the process is complicate and largely prolongs the surgery time.

Thus, the conventional positioning methods are difficult to practice and is time consuming, and have cumbersome operations, high cost, inaccurate positioning, and severe damages on the patient and the medical personals.

### SUMMARY OF THE INVENTION

In view of the above-described problems, it is one objective of the invention to provide an adhesive film with developing reference axis. The adhesive film is simple in structure, convenient for practice, and accurate in positioning.

To achieve the above objective, in accordance with one embodiment of the invention, there is provided an adhesive film comprising an upper surface comprising a developing reference axis.

In a class of this embodiment, the developing reference axis is provided with reference numbers.

In a class of this embodiment, the adhesive film comprises a plurality of layers of adhesive films. One or more layers of inner adhesive films are disposed beneath an uppermost adhesive film. Each of the inner adhesive films comprises an upper surface comprising a reference axis being the same as the developing reference axis arranged on the uppermost adhesive film. The reference axis arranged on each of the inner adhesive films superposes with one another.

In a class of this embodiment, the reference axis arranged on each of the inner adhesive films comprises reference numbers being the same as the reference numbers of the uppermost adhesive film.

In a class of this embodiment, the developing reference axis of the uppermost adhesive film is in a grid structure formed by arranging transverse lines and vertical lines at equal intervals between each other.

In a class of this embodiment, the reference axis arranged on each inner adhesive film is the same as the developing reference axis arranged on the uppermost adhesive film and is in a grid structure formed by arranging transverse lines and vertical lines at equal intervals between each other.

In a class of this embodiment, vertical lines and/ or transverse lines of the developing reference axis are developing lines in deferent shapes arranged at interval. A grid spacing of the developing reference axis is between 1 and 5 mm

In a class of this embodiment, the developing reference axis is formed by arranging points at equal intervals in a plurality of transverse lines and vertical lines.

Advantages of the invention are summarized as follows:

1) The adhesive film of the invention is provided with the developing reference axis, by which, the focus can be accurately positioned within one-step operation, so that the exposure duration and times of the radioactive rays on the patients and the medical personals decrease, so does the hazard of the radioactive rays on the patients and the medical personals. 2) The adhesive film is superior to the conventional method in that once the adhesive film is stuck to the focus and is positioned, the positioned points will not shade or fade away; thus, the accurate positioning is achieved, thereby ensuring the accuracy in subsequent surgery or nursing. 3) The positioned points on the adhesive film do not fade away so as not to probe or position repeatedly in the surgery and avoid corresponding injury on the patients. 4) The adhesive film has a wide application and is not restricted by the somatotype of the patients. 5) The adhesive film largely decreases the times for taking X-ray images, thereby saving the examination fee for the patients. 6) The adhesive film largely shortens the operation time in the surgery and the nursing, thereby improving the efficiency. 7) The adhesive film largely decreases the anesthetic intake of the patients. 8) The adhesive film is capable of protecting the surgical environment, avoiding cross infection of incisions, achieving the one-step positioning in the surgery, and largely simplifying the necessary environment for the surgery.

### BRIEF DESCRIPTION OF THE DRAWINGS

The invention is described hereinbelow with reference to the accompanying drawings, in which:

FIG. 1 is a structure diagram of an adhesive film comprising a grid type developing reference axis in accordance with one embodiment of the invention;

FIG. 2 is a structure diagram of a plurality of layers of adhesive films comprising a grid type reference axis in accordance with one embodiment of the invention;

FIG. 3 is a structure diagram of a first inner adhesive film comprising a grid type reference axis in accordance with one embodiment of the invention;

FIG. 4 is a structure diagram of an adhesive film comprising a grid type reference axis formed by lines of different shapes in accordance with one embodiment of the invention;

FIG. 5 is a structure diagram of an adhesive film comprising a point type reference axis in accordance with one embodiment of the invention; and

FIG. 6 is a structure diagram of a first inner adhesive film comprising a point type reference axis in accordance with one embodiment of the invention.

### DETAILED DESCRIPTION OF THE EMBODIMENTS

For further illustrating the invention, experiments detailing an adhesive film are described below. It should be noted that the following examples are intended to describe and not to limit the invention.

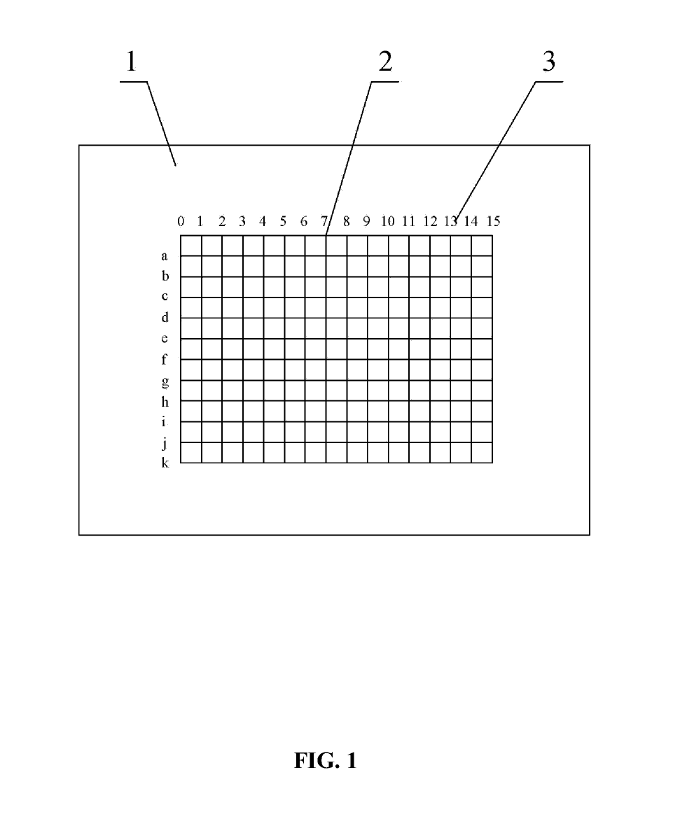

As shown in FIG. 1, a clinical adhesive film comprises an uppermost adhesive film 1 comprising an upper surface. A developing reference axis 2 is arranged on the upper surface of the uppermost adhesive film 1. The developing reference axis 2 is in the form of grids or points.

The developing reference axis 2 is in the grid structure formed by arranging transverse lines and vertical lines at equal intervals between each other. A grid spacing of the grid structure of the developing reference axis 2 is between 1 and 5 mm

For accurate positioning, such as positioning on hands and feet in arthrosteopedic surgery, a preferable grid spacing is between 1 and 3 mm

For conventional positioning, a preferable grid spacing is between 3 and 5 mm

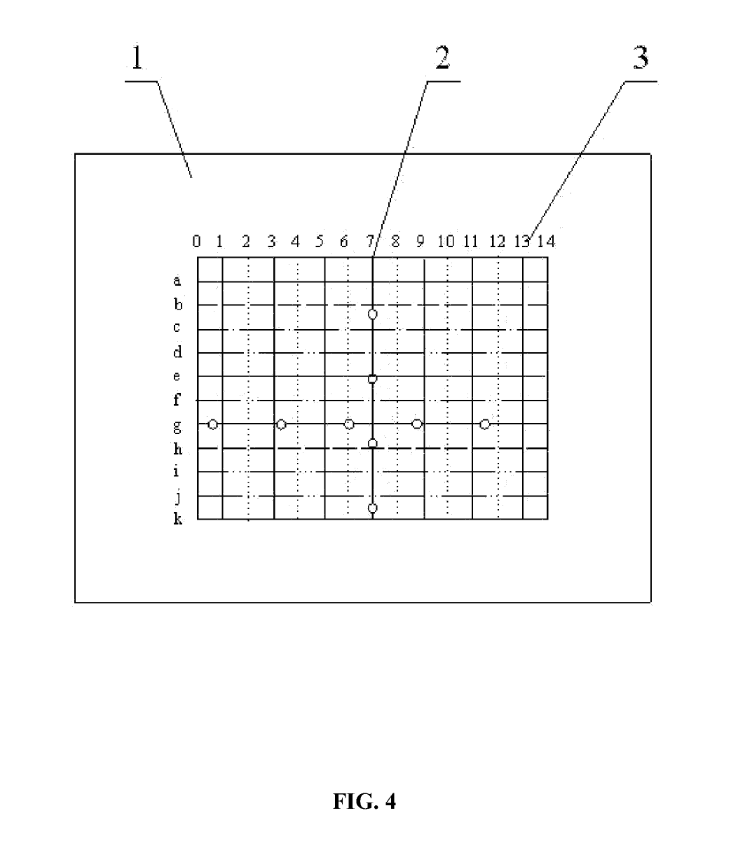

As shown in FIG. 4, the grid structure of the developing reference axis 2 is formed by arranging vertical lines and/ or transverse lines of different shapes at intervals.

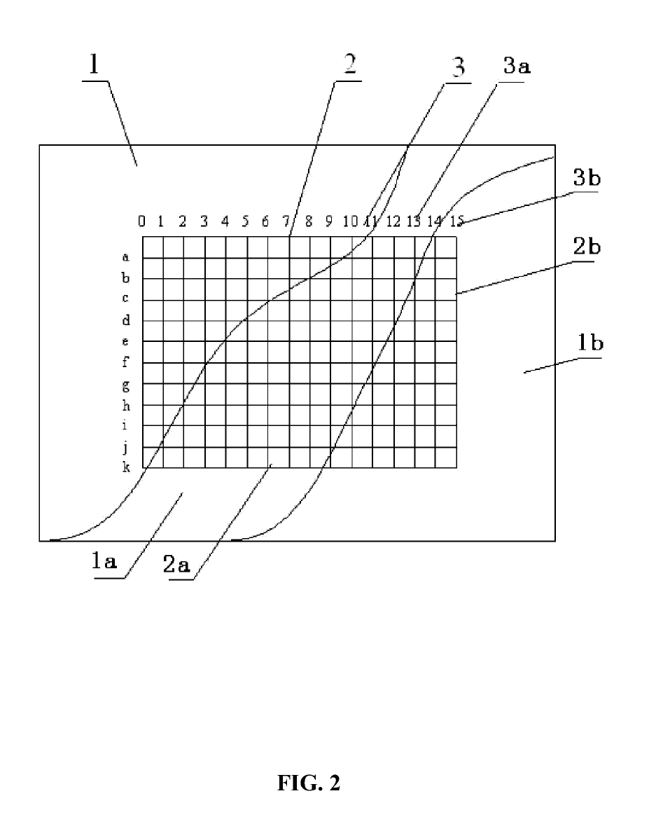

As shown in FIGS. 1-3, for sterilization treatment after positioning in surgery or other operations, the adhesive film of the invention comprises a plurality of layers of adhesive films attached to one another in the following order: the uppermost adhesive film 1, a first inner adhesive film 1a, a second inner adhesive film 1b . . . An upper surface of each of the first inner adhesive film 1a, the second inner adhesive film 1b . . . is provided with a reference axis 2a, 2b . . . being the same as the developing reference axis 2 of the uppermost adhesive film 1. The reference axis of each layers of adhesive films superposes one another. Thus, when the positioning is completed, it only requires to remove the uppermost adhesive film, because the inner adhesive films are in an aseptic state. Thus, the process of sterilization can be saved.

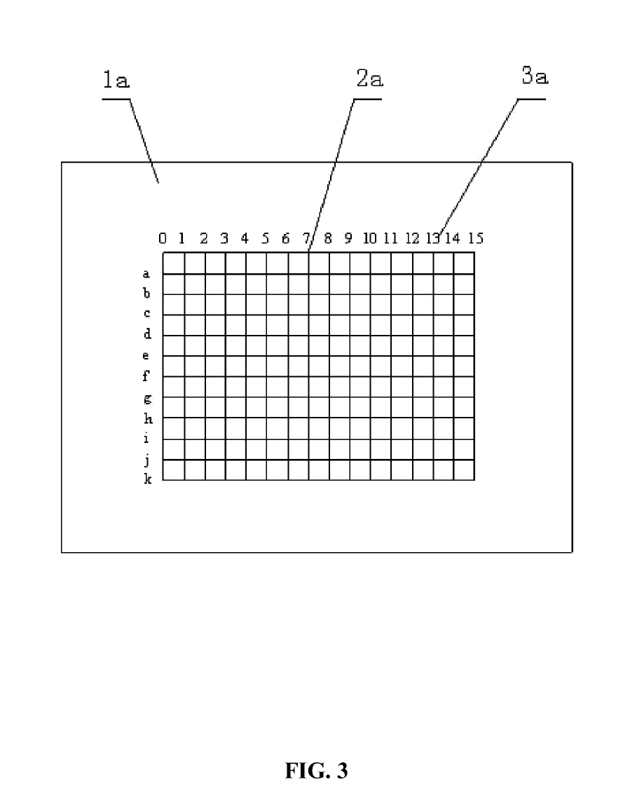

FIG. 3 is a structure diagram of the first inner adhesive film 1a. The grid type reference axis of each of the second inner adhesive film 1b and other inner adhesive films beneath are the same as those of the uppermost adhesive film 1 and the first inner adhesive film 1a.

The reference axis 2, 2a, 2b . . . of each layer can be printed by a developing agent, or a non-developing agent. The uppermost adhesive film 1 is commonly used for examination, thereby being provided with a developing reference axis. When repeated positioning is required, the reference axis 2a, 2b . . . of each inner adhesive film is printed by the developing agent. When a second positioning is not required, for example in radiotherapy operation, the reference axis 2a, 2b . . . of each inner adhesive film is printed by the non-developing agent. Thus, during the therapy, the reference axis 2a, 2b . . . of inner adhesive films will not shade the radiation or influence the therapy effect. Thus, the developing reference axis or the non-developing reference axis can be flexibly used as needed.

For convenient positioning, the reference axis 2, 2a, 2b . . . of each layer of the adhesive films is provided with reference numbers 3, 3a, 3b . . . The reference numbers 3, 3a, 3b . . . are numbers, alphabets, or a combination thereof. During positioning, doctors are able to accurately position the objective within the focus or the surgical field according to the readings of the reference numbers. Also, the reference numbers 3a, 3b . . . of the reference axis 2a, 2b . . . of the inner adhesive films are the same as the reference numbers 3 of the uppermost adhesive film 1.

### EXAMPLE 1

### Grid-Type Adhesive Film: As Shown in FIGS. 1-3

The adhesive film comprises a plurality of layers of adhesive films. An upper surface of an uppermost adhesive film 1 is provided with a developing reference axis 2. A first inner adhesive film 1a, a second inner adhesive film 1b . . . are arranged beneath the uppermost adhesive film 1. Each of the inner adhesive films 1a, 1b . . . comprises an upper surface comprising a reference axis 2a, 2b . . . being the same as the developing reference axis arranged on the uppermost adhesive film. The uppermost reference axis 2 of the uppermost adhesive film superposes the reference axis 2a, 2b . . . arranged on each of the inner adhesive films. The reference axis 2, 2a, 2b . . . of each layer can be printed by a developing agent, or a non-developing agent. The developing reference axis 2 of the uppermost adhesive film 1 is provided with reference numbers 3. The inner adhesive films are also provided with reference numbers 3a, 3b . . . being the same as the reference numbers 3 of the uppermost adhesive film.

The developing reference axis 2 of the uppermost adhesive film 1 and the reference axis 2a, 2b . . . of each inner adhesive film are in grid structure formed by arranging vertical lines and transverse lines at equal intervals. A grid spacing of the developing reference axis is between 3 and 5 mm

The adhesive film is capable of accurately positioning the focus.

As shown in FIG. 4, an improvement of the adhesive film is made for fast and accurately positioning the focus. The developing reference axis 2 of the uppermost adhesive film 1 and the reference axis 2a, 2b . . . of each inner adhesive film are formed by lines of different shapes. As shown in FIG. 4, the lines that form the grid are dash lines, dot-and-dash lines, double-dotted lines, or dotted lines.

### EXAMPLE 2

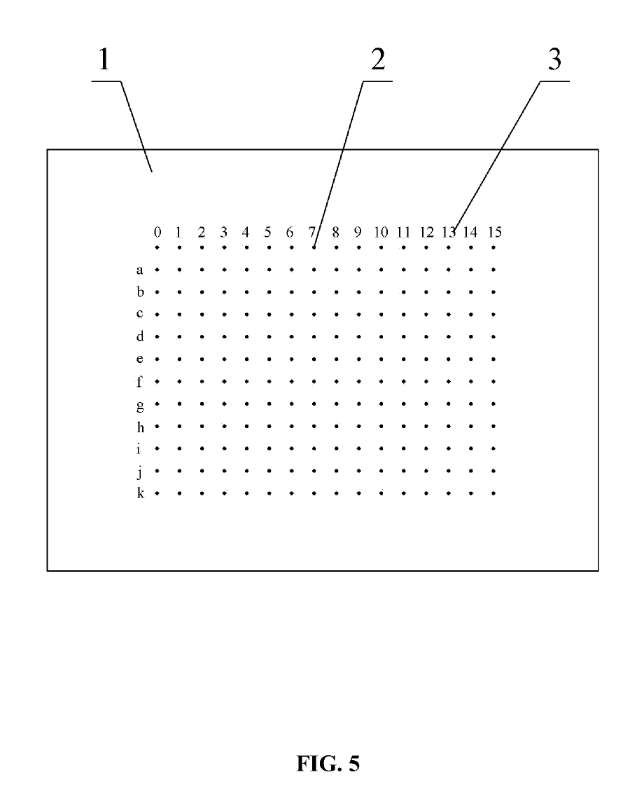

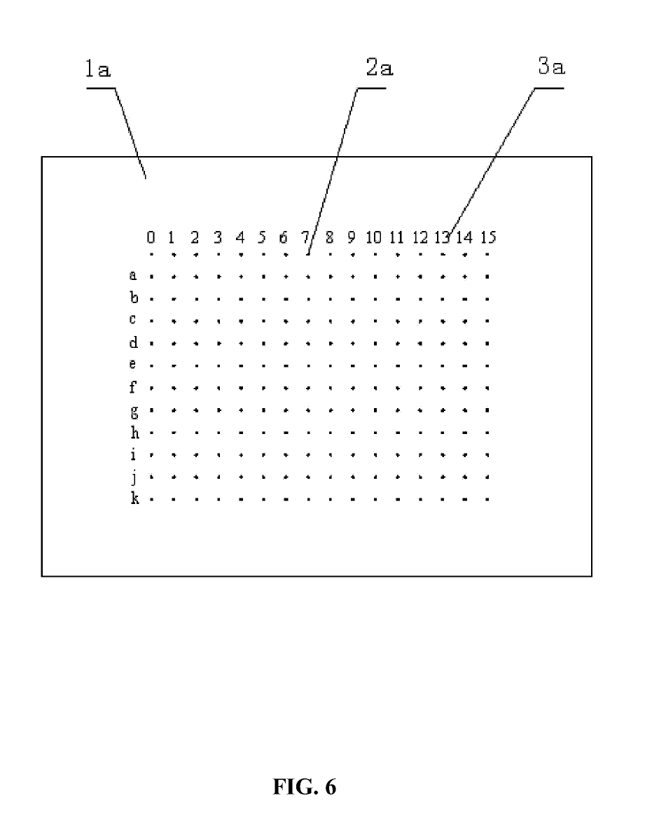

### Point-Type Adhesive Film: As Shown in FIGS. 5-6

The adhesive film comprises a plurality of layers of adhesive films, what is different from Example 1 is that the reference axis of each adhesive film is formed by arranging points at equal intervals in vertical lines and transverse lines.

The points are circle points, square points, triangle points, trapezoid points, or heart-shape points.

The uppermost adhesive film is provided with the developing reference axis for the purpose of examination. The inner adhesive films are provided with the developing reference axis or the non-development axis according to different use: for examination purpose, the corresponding inner adhesive film is provided with the developing reference axis; and for radiotherapy purpose, the corresponding inner adhesive film is provided with the non-developing reference axis. The number of the layers of the inner adhesive films is between 1 and 3.

The method for using the adhesive film of the invention is as follows: determine an approximate area of the focus according to the illness of the patient, and stick the adhesive film to the corresponding skin area. Take an image of the focus by using the X-ray so as to display the focus as well as the reference axis 2 of the uppermost adhesive film 1 on the X-ray image. Accurately positioning the focus according to the area reading of the reference axis 2. After positioning the focus, the positioned points remain on the reference axis 2 of the uppermost adhesive film 1. In subsequent therapy or surgery, remove the uppermost adhesive film 1, and position the focus by using the inner adhesive films, so that the sterilization treatment is not required. Comparisons of the influence on medical personals between a conventional positioning method and the method by using the adhesive film of the invention are summarized in Table 1.

TABLE 1
Comparisons of influence on medical personals between conventional
positioning methods and the method by using the adhesive film of the invention

| | Average | | | | | |
|---|---|---|---|---|---|---|
| | times for | | | | Average | |
| | using a | Average | Average | Average | incidence | Average |
| | perspective | exposure | exposure | working | of adverse | leukocyte |
| | device | dose | time | time | reactions | counting |
| Conventional | >5 | 0.138 | 2.5 s | 40 min | >15% | <4.0 × 10⁹/L |
| positioning | | millisievert | | | | |
| group | | | | | | |
| Developing | <2 | 0.0552 | 1 s | 15 min | <6% | 4.0-10.0 × 10⁹/L |
| positioning | | millisievert | | | | |
| group | | | | | | |

Comparisons of the influence on patients between a conventional positioning method and the method by using the adhesive film of the invention are summarized in Table 2.

TABLE 2
Comparisons of influence on patients between conventional positioning
methods and the method by using the adhesive film of the invention

| | Average | | | | | Average | |
|---|---|---|---|---|---|---|---|
| | times for | | | | Average | incidence | |
| | using a | Average | Average | Average | increased | of | Average |
| | perspective | exposure | exposure | surgery | anesthetic | adverse | leukocyte |
| | device | dose | time | duration | intake | reactions | counting |
| Conventional | >10 | 0.276 | 5 s | 40 min | >30% | >20% | <4.0 × 10⁹/L |
| positioning | | millisievert | | | | | |
| group | | | | | | | |
| Developing | <4 | 0.1104 | 2 s | 15 min | <10% | <8% | 4.0-10.0 × |
| positioning | | millisievert | | | | | 10⁹/L |
| group | | | | | | | |

From Tables 1-2, compared with the conventional positioning methods, the method by using the adhesive film of the invention can largely decrease the radiotherapy times, the exposure dose and time, the surgery duration, and the anesthetic intake. The influence of the radioactive rays on the medical personals and the patients are largely weakened.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An adhesive film, comprising an upper surface comprising a developing reference axis (2).

2. The adhesive film of claim 1, wherein the developing reference axis (2) is provided with reference numbers (3).

3. The adhesive film of claim 2, wherein
the adhesive film comprises an uppermost adhesive film and a plurality of layers of inner adhesive films (1a, 1b . . . );
the inner adhesive films (1a, 1b . . . ) are disposed beneath the uppermost adhesive film;
each of the inner adhesive films comprises an upper surface comprising a reference axis (2a, 2b . . . ) being the same as the developing reference axis (2) arranged on the uppermost adhesive film; and
the reference axis (2a, 2b . . . ) arranged on each of the inner adhesive films superposes with one another.

4. The adhesive film of claim 3, wherein the reference axis (2a, 2b . . . ) arranged on each inner adhesive film (1a, 1b . . . ) is provided with reference numbers (3a, 3b . . . ).

5. The adhesive film of claim 1, wherein the developing reference axis (2) is in a grid structure formed by arranging transverse lines and vertical lines at equal intervalss between each other.

6. The adhesive film of claim 3, wherein the reference axis (2a, 2b . . . ) arranged on each inner adhesive film is in a grid structure formed by arranging transverse lines and vertical lines at equal intervals between each other.

7. The adhesive film of claim 4, wherein the reference axis (2a, 2b . . . ) arranged on each inner adhesive film is in a grid structure formed by arranging transverse lines and vertical lines at equal intervals between each other.

8. The adhesive film of claim 5, wherein vertical lines and/ or transverse lines of the developing reference axis (2) arranged on the uppermost adhesive film are developing lines in deferent shapes arranged at interval.

9. The adhesive film of claim 7, wherein vertical lines and/ or transverse lines of the developing reference axis (2) arranged on the uppermost adhesive film are developing lines in deferent shapes arranged at interval.

10. The adhesive film of claim 5, wherein a grid spacing of the developing reference axis (2) arranged on the uppermost sticking layer is between 1 and 5 mm

11. The adhesive film of claim 8, wherein a grid spacing of the developing reference axis (2) arranged on the uppermost sticking layer is between 1 and 5 mm

12. The adhesive film of claim 1, wherein the developing reference axis (2) is formed by arranging points at equal intervals in a plurality of transverse lines and vertical lines.

13. The adhesive film of claim 3, wherein the developing reference axis (2) of the uppermost adhesive film is formed by arranging points at equal intervals in a plurality of transverse lines and vertical lines.

14. The adhesive film of claim 4, wherein the developing reference axis (2) of the uppermost adhesive film is formed by arranging points at equal intervals in a plurality of transverse lines and vertical lines.

15. The adhesive film of claim 13, wherein a distance between two adjacent points of the developing reference axis (2) of the uppermost adhesive film is between 3 and 5 mm

16. The adhesive film of claim 15, wherein a distance between two adjacent points of the developing reference axis (2) of the uppermost adhesive film is between 3 and 5 mm

17. The adhesive film of claim 5, wherein a grid spacing of the developing reference axis (2) of the uppermost adhesive film is between 1 and 2 mm

18. The adhesive film of claim 5, wherein a grid spacing of the developing reference axis (2) of the uppermost adhesive film is between 3 and 5 mm

19. The adhesive film of claim 3, wherein between 1 and 3 layers of the inner adhesive films are provided.

20. The adhesive film of claim 3, wherein at least one reference axis of the inner adhesive films is non-developing reference axis.
